(19) United States
(12) Patent Application Publication
(10) Pub. No.: US 2025/0339442 A1
(43) Pub. Date: Nov. 6, 2025

(54) METHODS OF TREATMENT USING A DUAL SPECIFICITY TYROSINE-PHOSPHORYLATION-REGULATED KINASE 1A (DYRK1A) INHIBITOR

(71) Applicant: Fresh Tracks Therapeutics, Inc., Boulder, CO (US)

(72) Inventors: Monica LUCHI, Boulder, CO (US); Deepak CHADHA, Boulder, CO (US); Nancy SERETTA, Boulder, CO (US)

(21) Appl. No.: 18/865,649

(22) PCT Filed: May 12, 2023
(86) PCT No.: PCT/US2023/022085

§ 371 Date: Nov. 13, 2024

Related U.S. Application Data
(60) Provisional application No. 63/364,679, filed on May 13, 2022.

(51) Int. Cl. A61K 31/5377

(52) U.S. Cl. CPC A61K 31/5377

(57) ABSTRACT

The present invention relates to methods, compositions, and unit dosage forms useful for treating an inflammatory disease, disorder, or condition in a patient.

### CROSS-REFERENCE TO RELATED APPLICATIONS

The application claims the benefit of priority to U.S. Provisional Appl. No. 63/364,679, filed May 13, 2022, the entirety of which is herein incorporated by reference.

### TECHNICAL FIELD OF THE INVENTION

The present invention relates to methods useful for inhibiting DYRKIA activity. The present invention relates to methods useful for selectively inhibiting DYRKIA activity to treat, for example, inflammatory conditions.

### BACKGROUND OF THE INVENTION

Autoimmune disease occurs when the immune system attacks self-molecules as a result of a breakdown of immunologic tolerance to autoreactive immune cells. Autoimmune disorders result from either congenital or acquired defects in central or peripheral immune tolerance. Many autoimmune disorders have been strongly associated with genetic, infectious, and/or environmental predisposing factors. Comprising multiple disorders and symptoms ranging from organ-specific to systemic, autoimmune diseases include insulin-dependent diabetes mellitus, rheumatoid arthritis, systemic lupus erythematosus, scleroderma, thyroiditis, and multiple sclerosis. A genetic propensity may underlie the development of most such disorders, but an external trigger may be required for the eventual development of the autoimmune disease. The development of pharmacologic agents that may modulate the immune system to treat such disorders has progressed over the last 60 years.

The appropriate and balanced differentiation of naïve CD4+ T helper (Th) cells into either proinflammatory effector subsets, such as Th1 and Th17 cells, or anti-inflammatory subsets, largely represented by regulatory T (Treg) cells, is an important determinant of immune homeostasis, dysregulation of which underlies the pathology of inflammatory diseases and cancer. It was demonstrated that DYRKIA can regulate T cell differentiation, in particular Th17 cells differentiation, which play an important role in the progression of inflammatory autoimmune diseases. Therefore, selective inhibition of DYRKIA can be developed as immunomodulatory intervention by inhibiting the population of Th17 cells that promotes autoimmunity and inflammation. This differentiation to Th17 requires expression of cytokines, such as IL-6, IL-23, and IL-21, through phosphorylation of STAT3. Compound A has been shown to reduce phosphorylation of STAT3 by inhibiting DYRKIA under inflammatory conditions induced by lipopolysaccharides.

There is urgent and compelling unmet medical need for more effective treatments for diseases, disorders or conditions associated with DYRKIA.

### SUMMARY OF THE INVENTION

The present invention provides, inter alia, methods of treating an inflammatory disease, disorder, or condition comprising administering to a patient in need thereof a therapeutically effective amount of (4-((4-(ethylamino)-3-(trifluoromethyl)-1H-pyrrolo [2,3-b]pyridin-6-yl)amino)-3-methoxyphenyl)(4-morpholinopiperidin-1-yl)methanone (Compound A) or a pharmaceutically acceptable salt or composition thereof.

In some embodiments, the inflammatory disease, disorder, or condition is a chronic inflammatory skin condition. For instance, in some embodiments, the inflammatory disease, disorder, or condition is an autoimmune diseases such as rheumatoid arthritis (RA), ulcerative colitis (UC), Crohn's disease, psoriasis, and atopic dermatitis (AD). In some embodiments, the inflammatory disease is characterized by intense itch, xerosis, and acute (erythematous papules, vesicles, edema, exudation, crusting), subacute, and chronic (scaly, erythematous papules and plaques, lichenification, excoriations, fissuring) eczematous skin lesions.

In some embodiments, the present invention further provides a composition comprising Compound A, or a pharmaceutically acceptable salt thereof.

In some embodiments, the present invention further provides a unit dosage form comprising Compound A, or a pharmaceutically acceptable salt thereof.

### BRIEF DESCRIPTION OF THE DRAWINGS

FIG. 1 depicts a diagram of the Part 1 study described in Example 1.

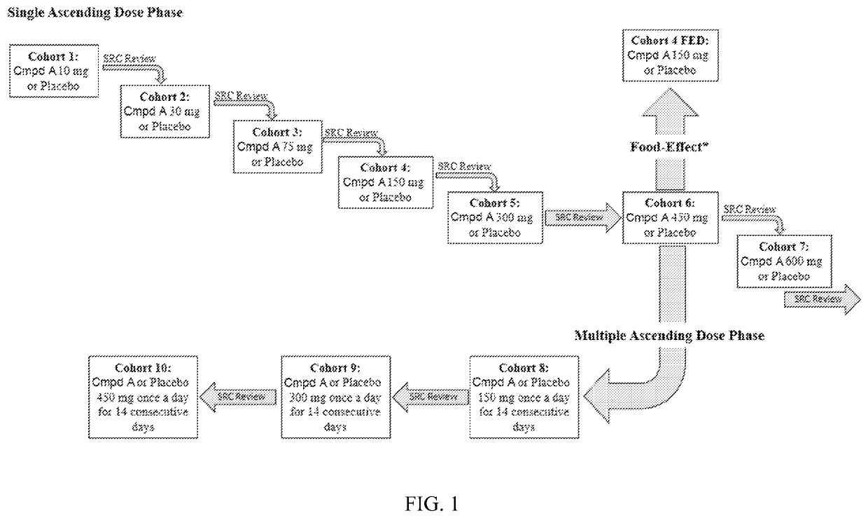

FIG. 2 depicts a diagram of the Part 2 study described in Example 1.

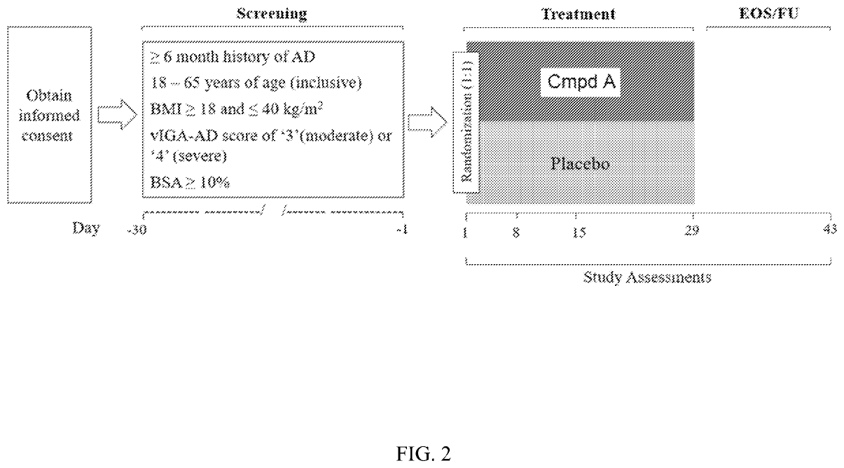

### DETAILED DESCRIPTION OF THE INVENTION

### 1. General Description of Certain Embodiments of the Invention

Atopic dermatitis (AD) is a common chronic inflammatory skin condition that affects both children and adults. It is characterized by intense itch, xerosis, and acute (erythematous papules, vesicles, edema, exudation, crusting), subacute, and chronic (scaly, erythematous papules and plaques, lichenification, excoriations, fissuring) eczematous skin lesions.

Occurrence of AD is most common in early infancy and childhood, with onset occurring in 45% during their first 6 months of life, 60% during their first year, and 90% affected before the age of 5. Onset typically occurs in children and can improve in adulthood; however, late onset can also occur. 5 Subjects can experience spontaneous disease remission later in adolescence but up to 50% will live with AD throughout adulthood. AD is estimated to impact approximately 15%-20% of children and 1%-3% of adults globally. Most subjects with AD present symptoms of mild to moderate severity.

Associated with AD are various other health-related comorbidities that can further negatively impact a subject's quality of life (QoL). An individual with AD may be predisposed to higher risk of other atopic disorders, including food allergies, allergic conjunctivitis/rhinitis, and asthma (atopic march). Chronic pruritus and inflammation/pain can lead to sleep disturbances and mental health symptoms. Subjects with AD are at higher risk for multiple neuropsychiatric disorders, including attention deficit (hyperactivity) disorder, depression, and suicidal ideation, speech disorders in childhood, headaches, and seizures. There are also cardio-metabolic and musculoskeletal (osteoporosis, injuries, and fractures) comorbidities.

Emollients and topical therapies (including corticosteroids, calcineurin inhibitors, and crisaborole) are commonly used as first-line treatment for AD flare-ups. Phototherapy, or systemic anti-inflammatory agents are also routinely used. If topical treatments do not provide subjects with adequate and sustained relief from AD symptoms, systemic steroids or other systemic immunosuppressive agents such as cyclosporine, azathioprine, methotrexate and mycophenolate mofetil are prescribed. While these can be effective as temporary treatments of flare-ups, extended use has been associated with many potential side effects or AEs.

Two novel developments for treatment of AD include: dupilumab, the biologic agent approved for moderate to severe AD, and oral Janus kinase (JAK) inhibitors, which are in late-stage clinical development. Nevertheless, despite the recent progress in the field, there is further necessity for a safe and efficacious agent for the treatment of AD.

Compound A is a novel immune-modulator being developed for the treatment of autoimmune diseases such as rheumatoid arthritis, ulcerative colitis, Crohn's disease, psoriasis, and atopic dermatitis. It acts by selectively inhibiting the dual specificity tyrosine-phosphorylation-regulated kinase 1A (DYRKIA). The investigational drug consists of the active ingredient Compound A HCl salt in a hard gelatin capsule shell to be taken by oral administration (PO) at 3 strength levels (10 mg, 75 mg, or 150 mg).

As outlined above, autoimmune disease occurs when the immune system attacks self-molecules as a result of a breakdown of immunologic tolerance to autoreactive immune cells. Autoimmune disorders result from either congenital or acquired defects in central or peripheral immune tolerance. Many autoimmune disorders have been strongly associated with genetic, infectious, and/or environmental predisposing factors. Comprising multiple disorders and symptoms ranging from organ-specific to systemic, autoimmune diseases include insulin-dependent diabetes mellitus, rheumatoid arthritis, systemic lupus erythematosus, scleroderma, thyroiditis, and multiple sclerosis. A genetic propensity may underlie the development of most such disorders, but an external trigger may be required for the eventual development of the autoimmune disease. The development of pharmacologic agents that may modulate the immune system to treat such disorders has progressed over the last 60 years.

The appropriate and balanced differentiation of naïve CD4+ T helper (Th) cells into either proinflammatory effector subsets, such as Th1 and Th17 cells, or anti-inflammatory subsets, largely represented by regulatory T (Treg) cells, is an important determinant of immune homeostasis, dysregulation of which underlies the pathology of inflammatory diseases and cancer. It was demonstrated that DYRKIA can regulate T cell differentiation, in particular Th17 cells differentiation, which play an important role in the progression of inflammatory autoimmune diseases. Therefore, selective inhibition of DYRKIA can be developed as immunomodulatory intervention by inhibiting the population of Th17 cells that promotes autoimmunity and inflammation. This differentiation to Th17 requires expression of cytokines, such as IL-6, IL-23, and IL-21, through phosphorylation of STAT3. Compound A has been shown to reduce phosphorylation of STAT3 by inhibiting DYRKIA under inflammatory conditions induced by lipopolysaccharides.

In human embryonic kidney cells (HEK-293), Compound A has demonstrated its anti-inflammatory potential in peripheral blood mononuclear cells (PBMCs) from subjects with ulcerative colitis, and its impact on the differentiation of CD4+ T cells by reducing pro inflammatory cytokines in PBMCs and by inhibiting the differentiation of Th1 and Th17 cells. Compound A also enhanced the development of Treg cells.

### Nonclinical Program

The nonclinical program supporting Compound A is based on a totality of evidence including published data of studies evaluating Compound A and the active metabolite in mice and beagle dogs via PO routes.

Compound A was evaluated in nonclinical in vitro, ex vivo and in vivo studies. The in vivo studies were all conducted in accordance with good laboratory practice (GLP) standards. A series of pharmacology, safety pharmacology, and toxicology studies were conducted with Compound A.

The anti-inflammatory effect of Compound A in PBMCs from subjects with ulcerative colitis was studied. The expression of pro-inflammatory cytokines IL-1β, IL 6, IL-8, IL-10 and TNF-α was investigated using a cytometric bead array (CBA). Results showed statistically significant reduction of pro-inflammatory cytokines by Compound A, indicating that Compound A has effects on the modulation of PBMC phenotype of subjects with ulcerative colitis based on the decreased level of pro-inflammatory cytokines.

Th1 and Th17 cells are main players contributing to organ specific autoimmune disease and inflammation. Results demonstrated that Compound A may improve inflammation through the inhibition of Th1 and Th7 cells and enhancing the development of Treg cells, making it a potential therapeutic candidate for the treatment of inflammatory diseases.

The anti-inflammatory activity of Compound A in the imiquimod (IMQ)-induced psoriasis-like dermal inflammation in BALB/c mice was investigated. It was concluded that Compound A at 45 and 60 mg/kg PO twice daily (BID)×9 days had significantly (p<0.05) protective effects on IMQ-induced psoriasis-like inflammation, as evidenced by the improvement in ear swelling in IMQ induced psoriasis model in BALB/c mice.

The potential anti-inflammatory and anti-pruritic effects of Compound A were investigated in a house dust-induced atopic dermatitis model in mice. Oral treatment with 30 mg/kg/day Compound A once (QD) or BID×21 days markedly reduced IL-4, IL-13 and IL-17 levels in the skin in comparison with controls. Additionally, immunohistochemical staining with antibodies for distribution analysis of T cell subsets showed that Compound A decreased all subset levels in the skin both in comparison with vehicle controls and the positive control, abrocitinib.

Furthermore, the efficacy of Compound A in the Collagen-Induced Arthritis Model in DBA/1 mice was tested. Immunization with Type II Chicken Collagen led to the development of arthritis. In comparison with vehicle only treated animals, mice treated once daily with 60 mg/kg Compound A led to a reduction in arthritis index scores, paw measurements, and histopathology scores.

In some embodiments the present invention provides a method of treating an inflammatory disease, disorder, or condition in a patient in need thereof, comprising the step of administering to the patient a therapeutically effective amount of compound A, or a pharmaceutically acceptable salt thereof.

In some embodiments, the inflammatory disease, disorder, or condition is chronic inflammatory skin condition.

In some embodiments, the inflammatory disease, disorder, or condition is atopic dermatitis (AD).

In some embodiments, the inflammatory disease, disorder, or condition is characterized by intense itch, xerosis, and acute (erythematous papules, vesicles, edema, exudation, crusting), subacute, and chronic (scaly, erythematous papules and plaques, lichenification, excoriations, fissuring) eczematous skin lesions.

In some embodiments, the present invention provides a composition as described herein, comprising compound A or a pharmaceutically acceptable salt thereof.

In some embodiments, the present invention provides a unit dosage form as described herein, comprising compound A or a pharmaceutically acceptable salt thereof.

### 2. Definitions

As used herein, the term “Compound A” refers to (4-((4-(ethylamino)-3-(trifluoromethyl)-1H-pyrrolo[2,3-b]pyridin-6-yl)amino)-3-methoxyphenyl)(4-morpholinopiperidin-1-yl)methanone, of formula:

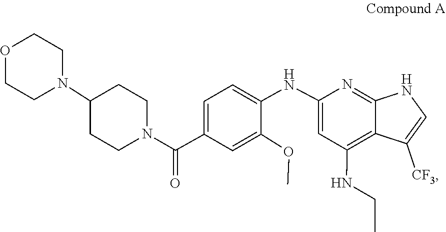 [SMILES: CCNc1cc(Nc2ccc(C(=O)N3CCC(N4CCOCC4)CC3)cc2OC)nc2[nH]cc(C(F)(F)F)c12]

or a pharmaceutically acceptable salt thereof. In some embodiments, Compound A is in the form of the HCl salt. U.S. Pat. No. 11,117,892 (“the '892 patent”) filed Sep. 19, 2019 as U.S. Pat. App. Serial No. U.S. Ser. No. 16/495,455 and published as U.S. Pat. App. Pub. No. U.S. 2020/0207756 (“the '756 publication”), the entirety of each is incorporated herein by reference, describe certain DYK1A inhibitor compounds, including Compound A. Compound A is designated as Example 57 in the '892 patent.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the terms “about” or “approximately” have the meaning of within 20% of a given value or range. In some embodiments, the term “about” refers to within 20%, 19%, 18%, 17%, 16%, 15%1, 14%1, 3% 12%, 11%, 10%, 9%, 8%, 7%, 6%, 5%, 4%, 3%, 2%, or 1% of a given value.

### 3. Description of Exemplary Methods and Uses

As used herein, the term “patient,” means an animal, preferably a mammal, and most preferably a human.

As used herein, the terms “treatment,” “treat,” and “treating” refer to reversing, alleviating, delaying the onset of, or inhibiting the progress of a disease, disorder, or condition or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the phrase “DYRKIA-mediated disease, disorder, or condition” refers to any disease or other deleterious condition in which DYRKIA is known to play a role. Accordingly, another embodiment of the present invention relates to treating or lessening the severity of one or more diseases in which DYRKIA is known to play a role.

Unless otherwise specified, it should be understood that when methods of the present invention described above and herein refer to administering Compound A, or a pharmaceutically acceptable salt thereof, said methods also contemplate administering a pharmaceutically acceptable composition comprising Compound A, or a pharmaceutically acceptable salt thereof.

As described above and herein, in some embodiments the present invention provides a method of modulating DYRKIA activity in a patient in need thereof, comprising administering to the patient a therapeutically effective amount of Compound A, or a pharmaceutically acceptable salt thereof.

In some embodiments, the present invention provides a method of inhibiting DYRKIA in a patient in need thereof, comprising administering to the patient a therapeutically effective amount of Compound A, or a pharmaceutically acceptable salt thereof.

In some embodiments, the present invention provides a method of treating an inflammatory disease, disorder, or condition in a patient in need thereof, comprising administering to the patient a therapeutically effective amount of Compound A, or a pharmaceutically acceptable salt thereof.

In some embodiments, the present invention provides a method of treating atopic dermatitis (AD) in a patient in need thereof, comprising administering to the patient a therapeutically effective amount of a pharmaceutically acceptable composition comprising Compound A, or a pharmaceutically acceptable salt thereof.

In some embodiments, the patient is diagnosed with atopic dermatitis (AD). In some embodiments, the patient has moderate atopic dermatitis (AD), for instance as defined by a Validated Investigator Global Assessment for Atopic Dermatitis [vIGA-AD]score of ‘3’. In some embodiments, the patient has severe atopic dermatitis (AD), for instance as defined by a Validated Investigator Global Assessment for Atopic Dermatitis [vIGA-AD]score of ‘4’.

In some embodiments, the patient is experiencing or has experiences a symptom such as intense itch, xerosis, and acute (erythematous papules, vesicles, edema, exudation, crusting), subacute, and chronic (scaly, erythematous papules and plaques, lichenification, excoriations, fissuring) eczematous skin lesions.

In some embodiments, a patient does not have one or more of the exclusion criteria as set forth in the Examples included herein.

In some embodiments, a patient has one or more of the inclusion criteria as set forth in the Examples included herein.

### 4. Dosing

In some embodiments, a method of the present invention comprises administering to a patient in need thereof a therapeutically effective amount of compound A, or pharmaceutically acceptable salt thereof, wherein the therapeutically effective amount comprises a total daily dose of about 1 mg to about 2000 mg, or about 1 mg to about 1900 mg, or about 1 mg to about 1800 mg, or about 1 mg to about 1700 mg, or about 1 mg to about 1600 mg, or about 1 mg to about 1500 mg, or about 1 mg to about 1400 mg, or about 1 mg to about 1300 mg, or about 1 mg to about 1200 mg, or about 1 mg to about 1100 mg, or about 1 mg to about 1000 mg, or about 1 mg to about 900 mg, or about 1 mg to about 800 mg, or about 1 mg to about 700 mg, or about 1 mg to about 600 mg, or about 5 mg to about 750 mg, or about 10 mg to about 750 mg, or about 10 mg to about 600 mg, or about 10 mg to about 550 mg, or about 10 mg to about 500 mg, or about 10 mg to about 450 mg, or about 10 mg to about 400 mg, or about 10 mg to about 350 mg, or about 10 mg to about 300 mg, or about 10 mg to about 250 mg, or about 10 mg to about 200 mg, or about 10 mg to about 150 mg, or about 10 mg to about 100 mg, or about 10 mg to about 75 mg, or about 10 mg to about 50 mg, or about 10 mg to about 30 mg, or about 10 mg to about 20 mg.

In some embodiments, a method of the present invention comprises administering to a patient in need thereof a therapeutically effective amount of Compound A, or pharmaceutically acceptable salt thereof, wherein the therapeutically effective amount comprises a total daily dose of about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a method of the present invention comprises administering to a patient in need thereof a therapeutically effective amount of Compound A, or pharmaceutically acceptable salt thereof, wherein the therapeutically effective amount comprises a total daily dose of about 10 mg, about 30 mg, about 75 mg, about 150 mg, about 300 mg about 450 mg, or about 600 mg.

In some embodiments, a method of the present invention comprises administering to a patient in need thereof a therapeutically effective amount of Compound A, or pharmaceutically acceptable salt thereof, wherein the therapeutically effective amount comprises a total daily dose between about 1 to about 100 mg/kg, or about 1 to about 90 mg/kg, or about 1 to about 80 mg/kg, or about 1 to about 70 mg/kg, or about 1 to about 60 mg/kg, or about 1 to about 50 mg/kg, or about 1 to about 40 mg/kg, or about 1 to about 35 mg/kg, or about 1 to about 30 mg/kg, or about 1 to about 25 mg/kg, or about 1 to about 20 mg/kg, or about 1 to about 19 mg/kg, or about 1 to about 18 mg/kg, or about 1 to about 17 mg/kg, or about 1 to about 16 mg/kg, or about 1 to about 15 mg/kg, or about 1 to about 14 mg/kg, or about 1 to about 13 mg/kg, or about 1 to about 12 mg/kg, or about 1 to about 11 mg/kg, or about 1 to about 10 mg/kg, or about 1 to about 9 mg/kg, or about 1 to about 8 mg/kg, or about 1 to about 7 mg/kg, or about 1 to about 6 mg/kg, or about 1 to about 5 mg/kg, or about 1 to about 4 mg/kg, or about 1 to about 3 mg/kg. In some embodiments, a total daily dose is between about 2 mg/kg and about 40 mg/kg, or about 5 mg/kg and about 40 mg/kg, or about 10 mg/kg and about 40 mg/kg, or about 15 mg/kg and about 40 mg/kg, or about 20 mg/kg and about 40 mg/kg, or about 25 mg/kg and about 40 mg/kg, or about 30 mg/kg and about 40 mg/kg, or about 35 mg/kg and about 40 mg/kg of the patient's body weight per day.

In some embodiments, a total daily dose of Compound A, or pharmaceutically acceptable salt thereof, is administered as once a day (QD). In some such embodiments, a total daily dose is any of those described above and herein. In some such embodiments, a total daily dose is about 10 mg, about 30 mg, about 75 mg, about 150 mg, about 300 mg about 450 mg, or about 600 mg. In some such embodiments, a total daily dose is about 100 mg, about 125 mg, about 150 mg, about 175 mg about 200 mg, about 225 mg about 250 mg, or about 275 mg.

In some embodiments, a total daily dose is administered as two, three, or four doses in one day. In some such embodiments, each dose is identical. In some such embodiments, at least one dose is different from another dose. In some embodiments, a total daily dose is any of those described above and herein, wherein the dose is administered “BID”. In some embodiments, a total daily dose is any of those described above and herein, wherein the dose is administered “TID”. In some embodiments, a total daily dose is any of those described above and herein, wherein the dose is administered “QID”.

In some embodiments, a total daily dose is administered to a patient under fed conditions. In some such embodiments, a total daily dose is any of those described above and herein. In some such embodiments, a total daily dose is administered QD. In some such embodiments, a total daily dose is administered orally. In some embodiments, a total daily dose is administered daily for at least 1, 2, 3, 4, 5, 6, or 7 consecutive days. In some embodiments, a total daily dose is administered daily for a period of time deemed appropriate by one of skill in the medical arts.

In some embodiments, a total daily dose is administered to a patient under fasted conditions. In some such embodiments, a total daily dose is any of those described above and herein. In some such embodiments, a total daily dose is administered QD. In some such embodiments, a total daily dose is administered orally. In some such embodiments, the patient fasts for at least about 1, 2, 3, 4, 5, 6, 7, 8, 9, 10, 11, 12, 13, 14, 15, 16, 17, 18, 19, 20, 21, 22, 23, or 24 hours prior to administration. In some such embodiments, the patient fasts for at least about two to eight hours prior to administration. In some such embodiments, the patient fasts for about two hours, about four hours, or about eight hours prior to administration.

In some embodiments, the patient fasts for an amount of time after administration. For instance, in some embodiments a patient fasts for about 1, 2, 3, 4, 5, 6, 7, or 8 hours after administration.

In some embodiments, a total daily dose of about 1 mg to about 1000 mg of Compound A, or pharmaceutically acceptable salt thereof, is administered to a patient once a day under fasted conditions. In some embodiments, a total daily dose of about 1 mg to about 1000 mg of Compound A, or pharmaceutically acceptable salt thereof, is administered to a patient once a day under fed conditions.

In some embodiments, a total daily dose of about 10 mg to about 600 mg of Compound A, or pharmaceutically acceptable salt thereof, is administered to a patient once a day under fasted conditions. In some embodiments, a total daily dose of about 10 mg to about 600 mg of compound A, or pharmaceutically acceptable salt thereof, is administered to a patient once a day under fed conditions.

In some embodiments, a total daily dose of about 50 mg to about 300 mg of Compound A, or pharmaceutically acceptable salt thereof, is administered to a patient once a day under fasted conditions. In some embodiments, a total daily dose of about 50 mg to about 300 mg of compound A, or pharmaceutically acceptable salt thereof, is administered to a patient once a day under fed conditions.

In some embodiments, provided methods comprise administering to a patient in need thereof a therapeutically effective amount of Compound A, or pharmaceutically acceptable salt thereof, comprising administering Compound A daily, weekly, or monthly. In some embodiments, provided methods comprise administering to a patient in need thereof a therapeutically effective amount of Compound A, or pharmaceutically acceptable salt thereof, comprising administering Compound A at the same time each day. For instance, in some embodiments, Compound A is administered at the same time each morning. In some embodiments, Compound A is administered at the same time each evening.

In some embodiments, a method of the present invention comprises administering Compound A or a unit dosage form thereof as described herein, wherein a Cmax of up to about 7000 ng/mL of Compound A in plasma is achieved. In some embodiments, the administration of Compound A or a pharmaceutically acceptable salt thereof (e.g., in a unit dose form as described herein) achieves a Cmax of up to about 6000 ng/mL of Compound A in plasma. In some embodiments, the administration of Compound A or a pharmaceutically acceptable salt thereof (e.g., in a unit dose form as described herein) achieves a Cmax of up to about 5000 ng/mL of Compound A in plasma. In some embodiments, the administration of Compound A or a pharmaceutically acceptable salt thereof (e.g., in a unit dose form as described herein) achieves a Cmax of up to about 4000 ng/mL of Compound A in plasma. In some embodiments, the administration of Compound A or a pharmaceutically acceptable salt thereof (e.g., in a unit dose form as described herein) achieves a Cmax of up to about 3000 ng/mL of Compound A in plasma.

In some embodiments, a Cmax of Compound A in plasma includes about 100 ng/mL, 200 ng/mL, 300 ng/mL, 400 ng/mL, 500 ng/mL, 600 ng/mL, 700 ng/mL, 800 ng/mL, 900 ng/mL, 1000 ng/mL, 1100 ng/mL, 1200 ng/mL, 1300 ng/mL, 1400 ng/mL, 1500 ng/mL, 1600 ng/mL, 1700 ng/mL, 1800 ng/mL, 1900 ng/mL, 2000 ng/mL, 2100 ng/mL, 2200 ng/mL, 2300 ng/mL. 2400 ng/mL, 2500 ng/mL, 2600 ng/mL, 2700 ng/mL, 2800 ng/mL, 2900 ng/mL, 3000 ng/mL, 3100 ng/mL, 3200 ng/mL, 3300 ng/mL, 3400 ng/mL, 3500 ng/mL, 3600 ng/mL, 3700 ng/mL, 3800 ng/mL, 3900 ng/mL, 4000 ng/mL, 4100 ng/mL, 4200 ng/mL, 4300 ng/mL, 4400 ng/mL, 4500 ng/mL, 4600 ng/mL, 4700 ng/mL, 4800 ng/mL, 4900 ng/mL, 5000 ng/mL, 5100 ng/mL, 5200 ng/mL, 5300 ng/mL, 5400 ng/mL, 5500 ng/mL, 5600 ng/mL, 5700 ng/mL, 5800 ng/mL, 5900 ng/mL, and 6000 ng/mL, or any range of Cmax created by using two of the aforementioned concentrations as endpoints.

In some embodiments, the present disclosure provides a method of administering Compound A to a patient in need thereof, comprising administering to said patient a therapeutically effective amount of Compound A or a pharmaceutically acceptable salt thereof (e.g., in a unit dose form as described herein), wherein an AUC of up to about 120,000 ng*h/mL of Compound A in plasma is achieved. In some embodiments, the administration of Compound A or a pharmaceutically acceptable salt thereof (e.g., in a unit dose form as described herein) achieves an AUC of up to about 110,000 ng*h/mL of Compound A in plasma. In some embodiments, the administration of Compound A or a pharmaceutically acceptable salt thereof (e.g., in a unit dose form as described herein) achieves an AUC of up to about 100,000 ng*h/mL of Compound A in plasma. In some embodiments, the administration of Compound A or a pharmaceutically acceptable salt thereof (e.g., in a unit dose form as described herein) achieves an AUC of up to about 40,000 ng*h/mL of Compound A in plasma.

In some embodiments, an AUC of Compound A in plasma includes about 1000 ng*h/mL, 2000 ng*h/mL, 3000 ng*h/mL, 4000 ng*h/mL, 5000 ng*h/mL, 6000 ng*h/mL, 7000 ng*h/mL, 8000 ng*h/mL, 9000 ng*h/mL, 10,000 ng/mL, 11,000 ng/mL, 12,000 ng*h/mL, 13,000 ng*h/mL, 14,000 ng*h/mL, 15,000 ng*h/mL, 16,000 ng*h/mL, 17,000 ng*h/mL, 18,000 ng*h/mL, 19,000 ng*h/mL, 20,000 ng/mL, 21,000 ng/mL, 22,000 ng*h/mL, 23,000 ng*h/mL, 24,000 ng*h/mL, 25,000 ng*h/mL, 26,000 ng*h/mL, 26,500 ng*h/mL, 27,000 ng*h/mL, 28,000 ng*h/mL, 29,000 ng*h/mL, 30,000 ng/mL, 31,000 ng/mL, 32,000 ng*h/mL, 33,000 ng*h/mL, 34,000 ng*h/mL, 35,000 ng*h/mL, 36,000 ng*h/mL, 37,000 ng*h/mL, 38,000 ng*h/mL, 39,000 ng*h/mL, 40,000 ng/mL, 41,000 ng/mL, 42,000 ng*h/mL, 43,000 ng*h/mL, 44,000 ng*h/mL, 45,000 ng*h/mL, 46,000 ng*h/mL, 47,000 ng*h/mL, 48,000 ng*h/mL, 49,000 ng*hi/mL, 50,000 ng/mL, 51,000 ng/mL, 52,000 ng*h/mL, 53,000 ng*h/mL, 54,000 ng*h/mL, 55,000 ng*h/mL, 56,000 ng*h/mL, 57,000 ng*h/mL. 58,000 ng*h/mL, 59,000 ng*h/mL, 60,000 ng/mL, 61,000 ng/mL, 62,000 ng*h/mL, 63,000 ng*h/mL, 64,000 ng*h/mL, 65,000 ng*h/mL, 66,000 ng*h/mL, 67,000 ng*h/mL, 68,000 ng*h/mL, 69,000 ng*h/mL, 70,000 ng/mL, 71,000 ng/mL, 72,000 ng*h/mL, 73,000 ng*h/mL, 74,000 ng*h/mL, 75,000 ng*h/mL, 76,000 ng*h/mL, 77,000 ng*h/mL, 78,000 ng*h/mL, 79,000 ng*h/mL, 80,000 ng/mL, 81,000 ng/mL, 82,000 ng/mL, 83,000 ng*h/mL, 84,000 ng*h/mL, 85,000 ng*h/mL, 86,000 ng*h/mL, 87,000 ng*h/mL, 88,000 ng*h/mL, 89,000 ng*h/mL, 90,000 ng/mL, 91,000 ng/mL, 92,000 ng/mL, 93,000 ng*h/mL, 94,000 ng*h/mL, 95,000 ng*h/mL, 96,000 ng*h/mL, 97,000 ng*h/mL, 98,000 ng*h/mL, 99,000 ng*h/mL, and 100,000 ng/mL, or any range of AUC created by using two of the aforementioned concentrations as endpoints.

In some embodiments, the present disclosure provides a method of administering Compound A to a patient in need thereof, comprising administering to said patient a therapeutically effective amount of Compound A or a pharmaceutically acceptable salt thereof (e.g., in a unit dose form as described herein), wherein a tmax of Compound A in plasma is from about 1 hrs to about 7 hrs. In some embodiments, the tmax of Compound A in plasma is from about 1 hrs to about 2 hrs, about 1 hrs to about 3 hrs, about 1 hrs to about 4 hrs, about 2 hrs to about 3 hrs, about 2 hrs to about 4 hrs, about 2 hrs to about 5 hrs, about 3 hrs to about 4 hrs, about 3 hrs to about 5 hrs, about 3 hrs to about 6 hrs, about 3 hrs to about 7 hrs, about 4 hrs to about 5 hrs, about 4 hrs to about 6 hrs, or about 4 hrs to about 7 hrs.

In some embodiments, the present disclosure provides a method of administering Compound A to a patient in need thereof, comprising administering to said patient a therapeutically effective amount of Compound A or a pharmaceutically acceptable salt thereof (e.g., in a unit dose form as described herein), wherein a t1/2 of Compound A in plasma is from about 5 hrs to about 30 hrs. In some embodiments, the t1/2 of Compound A in plasma is from about 5 hrs to about 10 hrs, about 5 hrs to about 15 hrs, about 5 hrs to about 20 hrs, about 5 hrs to about 25 hrs, about 10 hrs to about 15 hrs, about 10 hrs to about 20 hrs, about 10 hrs to about 25 hrs, about 15 hrs to about 20 hrs, about 15 hrs to about 25 hrs, about 15 hrs to about 30 hrs, about 20 hrs to about 25 hrs, or about 20 hrs to about 30 hrs.

In some embodiments, the present disclosure provides a method of administering Compound A to a patient in need thereof, comprising administering to said patient a therapeutically effective amount of Compound A or a pharmaceutically acceptable salt thereof (e.g., in a unit dose form as described herein), wherein a change in QT prolongation in the patient from baseline is achieved. In some embodiments, the mean change from baseline in QTcF in the patient is no greater than 20 ms. In some embodiments, the mean change from baseline in QTcF in the patient is no greater than 15 ms. In some embodiments, the mean change from baseline in QTcF in the patient is no greater than 10 ms. In some embodiments, the mean change from baseline in QTcF in the patient is no greater than 5 ms. In some embodiments, the mean change from baseline in QTcF in the patient returns to baseline and remained in the normal range after dosing cessation.

### 4. Unit Dosage Forms

In some embodiments, methods of the present invention comprise administering to a patient in need thereof a pharmaceutical composition comprising one or more unit doses of Compound A, or pharmaceutically acceptable salt thereof. In some such embodiments, a unit dose is about 10 mg to about 2000 mg, or about 10 mg to about 1000 mg, or about 10 mg to about 900 mg, or about 10 mg to about 800 mg, or about 10 mg to about 750 mg, or about 10 mg to about 700 mg, or about 10 mg to about 650 mg, or about 10 mg to about 600 mg, or about 10 mg to about 550 mg, or about 10 mg to about 500 mg, or about 10 mg to about 450 mg, or about 10 mg to about 400 mg, or about 10 mg to about 350 mg, or about 10 mg to about 300 mg, or about 10 mg to about 250 mg, or about 10 mg to about 200 mg, or about 10 mg to about 150 mg, or about 10 mg to about 100 mg. In some embodiments, a unit dosage form is about 10 mg, about 75 mg, or about 150 mg.

In some embodiments, a unit dosage form is of any of the above and is in the form of a capsule.

In some embodiments, a unit dose of Compound A, or pharmaceutically acceptable salt thereof, is administered orally once daily. In some such embodiments, a unit dose is any of those described above and herein.

### 5. Pharmaceutically Acceptable Compositions

In some embodiments, a method of the present invention comprises administering a composition comprising a therapeutically effective amount of Compound A, or a pharmaceutically acceptable salt thereof, and a pharmaceutically acceptable carrier, adjuvant, or vehicle. In certain embodiments, compositions for use in methods provided herein are formulated for administration to a patient in need of such composition, for instance for an inflammatory disorder. In some embodiments, such compositions are formulated for oral administration to a patient.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain along-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions for use in provided methods may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

In some embodiments, pharmaceutically acceptable compositions for use in provided methods are formulated for oral administration. Such formulations may be administered with or without food. In some embodiments, pharmaceutically acceptable compositions for use in provided methods are administered without food. In some embodiments, pharmaceutically acceptable compositions for use in provided methods are administered with food.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, c) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The compound can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Alternatively, pharmaceutically acceptable compositions for use in provided methods may be administered in the form of suppositories for rectal or vaginal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions for use in provided methods may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, pharmaceutically acceptable compositions for use in provided methods may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions for use in provided methods may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of Compound A that may be combined with the carrier materials to produce a composition in a single dosage form will vary depending upon the host treated and the particular mode of administration. In some embodiments, compositions for use in provided methods should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the age, body weight, general health, sex, diet, time of administration, rate of excretion, drug combination, and the judgment of the treating physician and the severity of the particular disease being treated.

As described above and herein, pharmaceutically acceptable compositions for use in provided methods can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

### List of Abbreviations

AD atopic dermatitis
AE adverse event
ALP alkaline phosphatase
ALT alanine aminotransferase
ANOVA analysis of variance
anti-HBc antibody to hepatitis B core antigen
aPTT activated partial thromboplastin time
AST aspartate aminotransferase
β-hCG β-human chorionic gonadotropin
BMI body mass index
BID twice daily
BP blood pressure
BSA body surface area
BUN blood urea nitrogen
CBA cytometric bead array
CONSORT Consolidated Standard of Reporting Trials
COVID-19 coronavirus disease—2019
CRA clinical research associate
CRO contract research organization
CRU clinical research unit
DLQI Dermatology Life Quality Index
DYRKIA dual specificity tyrosine-phosphorylation-regulated kinase 1A
EASI Eczema Area and Severity Index
EASI50 50% or greater improvement in Eczema Area and Severity Index
EAS175 75% or greater improvement in Eczema Area and Severity Index
ECG electrocardiogram
eCRF electronic case report form
EDC electronic data capture
EOT end of treatment
EOS end of study
ET early ternination
F bioavailability
FDA Food and Drug Administration
FIH first-in-human
FSH follicle-stimulating hormone
FU follow-up
GCP Good Clinical Practice
GGT gamma-glutamyl-transferase
GLP Good Laboratory Practice
HBsAg hepatitis B surface antigen
HBV hepatitis B virus
HCT hematocrit
HCV hepatitis C virus
HCV Ab hepatitis C virus antibody
HED human equivalent dose
HEENT head, eyes, ears, nose, throat
HEK human embryonic kidney
hERG human ether-a-go-go-related gene
Hgb hemoglobin
HIV human immunodeficiency virus
HPMC hydroxypropyl methylcellulose
HR heart rate
hs-CRP high-sensitivity C-reactive protein
IB investigator brochure
ICF informed consent form
ICH International Council for Harmonisation
IEC independent ethics committee
IHC immunohistochemistry
IL-33 interleukin-33
IMQ imiquimod
INR international normalized ratio
IRB institutional review board
IVIg intravenous immunoglobulin
IWRS Interactive web response system
LDH lactate dehydrogenase
LMW low molecular weight
LPS lipopolysaccharide
LS means least square means
MAD multiple ascending dose
MCH mean corpuscular hemoglobin
MCHC mean corpuscular hemoglobin concentration
MCV mean corpuscular volume
MDMA 3,4-Methylenedioxymethamphetamine
MedDRA Medical Dictionary for Regulatory Activities
mITT modified intent-to-treat
MMRM mixed model for repeated measures
MPV mean platelet volume
N/A not applicable
NC not calculable
nGLP non-Good Laboratory Practices
NOAEL no observed adverse effect level
NRS Numerical Rating Scale
NSAID nonsteroidal anti-inflammatory drug
OT oral temperature
PAD pharmacologically active dose
PBMC peripheral blood mononuclear cell
PCP phencyclidine
PD pharmacodynamic
PK pharmacokinetic
PLT platelets
PO per oral
POEM Patient-Oriented Eczema Measure
PP per-protocol
PPD purified protein derivative
PR PR interval
PT prothrombin time
PUVA psoralen-UV-A
QA quality assurance
QC quality control
QD once daily
QoL Quality of Life
QRS duration of ventricular muscle depolarization (ECG)
QTc corrected QT interval
QTcF Frederica's corrected QT intervals
QTcV Van de Water's corrected QT interval
RBC red blood cell (count)
REB research ethics board
RR respiratory rate
RT-PCR reverse transcription polymerase chain reaction
SAD single ascending dose
SAE serious adverse event
SAN sinoatrial node
SAP statistical analysis plan
SCORAD SCORing Atopic Dermatitis
SD standard deviation
SOC system organ class
SOP standard operating procedure
SRC safety review committee
TB tuberculosis
TEAE treatment-emergent adverse event
Th T helper
TK toxicokinetic
T_{reg} regulatory T cells
TSH thyroid-stimulating hormone
ULN upper limit of normal
UV ultraviolet
vIGA-AD Validated Investigator Global Assessment for Atopic Dermatitis
WBC white blood cell (count)
WHO world health organization
WOCBP women of childbearing potential

### Example 1: a Phase 1, Randomized, Double-Blind, Placebo-Controlled Study to Evaluate the Safety, Tolerability, Pharmacokinetics, Pharmacodynamics, and Preliminary Efficacy of Single and Multiple Ascending Doses of Compound a in Healthy Subjects and Subjects with Atopic Dermatitis

### Number of Subjects:

### Part 1:

Approximately 89 healthy subjects will be enrolled in this study and separated into a total of 10 cohorts;
Part 1A (single ascending dose [SAD]) will consist of 7 cohorts of 8 subjects, for a total of 56 subjects.
Part 1B (multiple ascending dose [MAD]) will consist of 3 cohorts of 11 subjects, for a total of 33 subjects.

Subjects who withdraw or are withdrawn from the study after dosing, for reasons other than safety and tolerability, may be replaced after consultation between the safety review committee (SRC) members. The total number of subjects dosed (including potential replacement subjects) will remain within a maximum of 10 subjects per cohort for Part 1A and 14 subjects per cohort for Part 1B.

Depending on safety and tolerability, additional cohorts may be added.

### Part 2:

Approximately 40 subjects with moderate to severe atopic dermatitis (AD) will be included in Part 2.

### Duration of Study, Confinement, and Washout:

### Part 1A:

The estimated duration per subject for Cohorts 1, 2, 3, and 5 to 7 is approximately 36 days, from screening through the follow-up period. Subjects will be confined from Day −1 until after the 96-hour post-dose blood draw on Day 5. Subjects will return to the clinic for the follow-up visit 7 days post-dose for additional blood draws and procedures.

The estimated duration per subject for Cohort 4 (food effect) is approximately 43 days, from screening through the follow-up period. Subjects will be confined from Day −1 until after the 96-hour post-dose blood draw on Day 5 of each period. There will be a washout period of at least 7 days between dosing of the fasting and the fed periods for Cohort 4, and the fed period is planned to occur after Cohort 5. If the washout period between doses is more than 7 days, subjects will return for a follow-up visit on Day 8. A follow-up visit will also be scheduled 7 days post-dose in period 2.

### Part 1B:

The estimated duration per subject is approximately 49 days, from screening through the follow-up period. Subjects will be confined from Day −1 until after the 96-hour post-last dose blood draw on Day 18. Subjects will return to the clinic for the follow-up visit 7 days post-last dose for additional blood draws and procedures.

### Part 2:

The estimated duration per subject of Part 2 is approximately 73 days, from screening through the follow-up period. Subjects in Part 2 will not be confined and will come to the study center on Days 1, 8, 15, 29, and 43 (follow-up visit).

### Investigational Products, Dosage, and Mode of Administration:

Investigational Drug: Compound A is a novel immune-modulator with a potential to treat autoimmune diseases such as rheumatoid arthritis, ulcerative colitis, Crohn's disease, psoriasis, and AD. The investigational drug, Compound A, is formulated for oral administration as capsules at doses of 10 mg, 75 mg, and 150 mg, to achieve appropriate dose levels per cohort as listed in the tables below.

Placebo: Capsules (of the same size and color as the investigational drug) will be administered as a matched oral administration.

### Part 1:

Part 1A (SAD—Cohorts 1 to 7): Subjects in each cohort will be randomized to receive a single oral dose of Compound A or matching placebo under fasting conditions (and under fed conditions for Cohort 4, Period 2) at the following dose levels:

| | | Planned | | | |
|---|---|---|---|---|---|
| | Number | Compound | | | |
| Co- | of | A dose | | Number of Compound | |
| hort | Subjects^{a} | levels | | A or Placebo capsules | Condition |
| 1 | 8 (6:2) | 10 | mg | 1 × 10 mg Compound A | Fasted |
| | | | | capsule or matching | |
| | | | | placebo capsule | |
| 2 | 8 (6:2) | 30 | mg | 3 × 10 mg Compound A | Fasted |
| | | | | capsules or matching | |
| | | | | placebo capsules | |
| 3 | 8 (6:2) | 75 | mg | 1 × 75 mg Compound A | Fasted |
| | | | | capsule or matching | |
| | | | | placebo capsule | |
| 4 | 8 (6:2) | 150 | mg | 1 × 150 mg Compound A | Period 1: |
| | | | | capsule or matching | Fasted |
| | | | | placebo capsule | Period 2: |
| | | | | | Fed |
| 5 | 8 (6:2) | 300 | mg | 2 × 150 mg Compound A | Fasted |
| | | | | capsules or matching | |
| | | | | placebo capsules | |
| 6 | 8 (6:2) | 450 | mg | 3 × 150 mg Compound A | Fasted |
| | | | | capsules or matching | |
| | | | | placebo capsules | |
| 7 | 8 (6:2) | 600 | mg | 4 × 150 mg Compound A | Fasted |
| | | | | capsule or matching | |
| | | | | placebo capsules | |

^{a}N (Compound A:placebo)

Part 1B (MAD—Cohorts 8 to 10): Subjects in each cohort will be randomized to receive multiple doses of Compound A or matching placebo at the following dose levels:

| | Number | Planned | Number of |
|---|---|---|---|
| | of | Compound A | Compound A or |
| Cohort | Subjects^{a} | dose levels | Placebo capsules |
| 8 | 11 (9:2) | 150 mg once a day | 1 × 150 mg Compound A |
| | | for 14 days | capsule or matching |
| | | | placebo capsule |
| 9 | 11 (9:2) | 300 mg once a day | 2 × 150 mg Compound A |
| | | for 14 days | capsules or matching |
| | | | placebo capsules |
| 10 | 11 (9:2) | 450 mg once a day | 3 × 150 mg Compound A |
| | | for 14 days | capsules or matching |
| | | | placebo capsules |

^{a}N (Compound A:placebo)

For each cohort of both Part 1A and Part 1B, a SRC (composed by at least the investigator, a medical monitor, and a medical sponsor representative) will review the safety and tolerability data, as well as available pharmacokinetic (PK) data, in order to make a decision regarding continuation of the study at the next prescribed dose level, decreasing the next dose level, repeating a dose level or to not evaluate any additional dosage, based on consideration of the clinical significance of several safety, tolerability and PK parameters. Some alterations from the currently outlined dose and/or dosing regimen may be performed, but the dose (or daily dose for Part 1B) to be administered in a given cohort will not exceed the one currently outlined in the protocol.

### Part 2:

Subjects will be randomized to a 1:1 ratio at baseline (Day 1) to receive oral Compound A or matching placebo capsules, for 28 days.

Part 2 will be initiated after completion of Part 1A and Part 1B and evaluation by the SRC of the available safety and PK data accrued in Part 1A and Part 1B. The dose of Compound A to be tested in Part 2 should not exceed the highest tolerable dose tested in Part 1B (MAD).

### Objectives and Endpoints:

### Part 1:

| | |
|---|---|
| OBJECTIVES | ENDPOINTS |

Primary

| | |
|---|---|
| To evaluate the safety and | Primary safety and tolerability endpoints: |
| tolerability of Compound A | Incidence and severity of TEAEs |
| capsules following oral | Change from baseline in vital signs, ECG, and safety |
| administration of single and | laboratory tests |
| multiple ascending doses in healthy | |
| subjects. | |

Secondary

| | |
|---|---|
| To characterize the PK profile of | Secondary PK endpoints: |
| Compound A and M3 metabolite in | Plasma PK parameters for Compound A and M3 |
| plasma following single and | metabolite will include: |
| multiple oral doses in healthy | Part 1A: AUC₀₋ₜ, AUC_{0-inf}, Cₘₐₓ, Residual Area, |
| subjects. | Tₘₐₓ, T_{1/2 el}, Kₑₗ, Cl/F, and V_{d}/F |
| To characterize the PK profile of | Part 1B (Day 1): AUC₀₋₂₄, Cₘₐₓ, and Tₘₐₓ |
| Compound A and M3 metabolite in | Part 1B (Day 14): AUC_{0-τ}, C_{max ss}, T_{max ss}, C_{trough}, |
| urine following a single oral dose in | AUC₀₋ₜ, AUC_{0-inf}, Residual Area, T_{1/2 el}, Kₑₗ, Clₛₛ/F, |
| healthy subjects. | and Vₛₛ/F |
| To evaluate the effect of food on the | Part 1A: Urine PK parameters for Compound A and |
| absorption of Compound A | M3 metabolite will include: |
| following a single oral dose in | Ae₀₋ₜ, Rₘₐₓ, T_{Rmax}, and Cl_{R} |
| healthy subjects. | |
| To characterize the PD profile of | Secondary PD endpoints: |
| Compound A in blood following | Changes from baseline in selected biomarkers in |
| multiple oral doses in healthy | PBMCs |
| subjects. | Relative changes from baseline in plasma log-scale |
| | cytokine levels |
| To describe the effect Compound A | Secondary safety endpoints: |
| on ECG parameters following single | Change-from-baseline HR, QTcF, PR and QRS |
| oral doses in healthy subjects.* | (ΔHR, ΔQTcF, ΔPR and ΔQRS); |
| | Placebo-corrected ΔHR, ΔQTcF, ΔPR and ΔQRS |
| | (ΔΔHR, ΔΔQTcF, ΔΔPR and ΔΔQRS); |
| | Categorical outliers for HR, QTcF, PR, QRS; |
| | Frequency of treatment emergent changes of T-wave |
| | morphology and U-wave presence |

*Cardiodynamic ECG evaluation will be performed on data from the SAD cohorts (Part 1A). If observed plasma concentrations of Compound A or metabolite are higher than expected, the cardiodynamic ECG evaluation may also be undertaken on data from the MAD cohorts (Part 1B).

| | |
|---|---|
| OBJECTIVES | ENDPOINTS |

Primary

| | |
|---|---|
| To evaluate the safety and | Primary safety and tolerability endpoints: |
| tolerability of Compound A | Incidence and severity of TEAEs |
| capsules administered orally for | Change from baseline in vital signs, ECG, and safety |
| 28 days to subjects with moderate to | laboratory tests |
| severe AD. | |

Secondary

| | |
|---|---|
| To evaluate the effect of Compound | Secondary PD endpoints: |
| A on skin biomarkers in subjects | Change from baseline in AD biomarkers in lesional |
| with moderate to severe AD. | and nonlesional skin, including but not limited to: |
| | General inflammation, hyperplasia, Th2 |
| | response, Th22 response |
| | Change from baseline in T-lymphocyte subset |
| | populations |
| | Changes from baseline in T-cell and dendritic cell |
| | markers |
| To evaluate the PK of Compound A | Secondary PK endpoint: |
| and M3 metabolite in plasma in | Plasma Compound A and M3 concentrations |
| subjects with moderate to severe | |
| AD. | |

Exploratory

| | |
|---|---|
| To evaluate the preliminary efficacy | Exploratory efficacy endpoints: |
| of Compound A capsules | Change and percent change from baseline EASI at |
| administered orally for 28 days to | Weeks 1, 2, and 4 |
| subjects with moderate to severe | Proportion of subjects achieving a EASI50 score |
| AD. | from baseline at Weeks 1, 2, and 4 |
| | Proportion of subjects achieving a EASI75 score |
| | from baseline at Weeks 1, 2, and 4 |
| | Change from baseline in vIGA-AD score at |
| | Weeks 1, 2, and 4 |
| | Proportion of subjects with vIGA-AD score of |
| | Clear (0) or Almost Clear (1) with a 2-point |
| | improvement from baseline at Weeks 1, 2, and 4 |
| | Change and percent change from baseline in BSA |
| | involvement at Week 1, 2, and 4 |
| | Change from baseline in weekly mean of the daily |
| | 24-hour pruritus NRS score at Weeks 1, 2, 3, and 4 |
| | Among subjects with a baseline pruritus NRS |
| | score ≥4, proportion of subjects with ≥4-point |
| | improvement in weekly mean of the daily 24-hour |
| | pruritus NRS score from baseline at Weeks 1, 2, 3, |
| | and 4 |
| | Among subjects with a baseline pruritus NRS |
| | score ≥3, proportion of subjects with ≥3-point |
| | improvement in weekly mean of the daily 24-hour |
| | pruritus NRS score from baseline at Weeks 1, 2, 3, |
| | and 4 |
| | Change from baseline in SCORAD at Weeks 1, 2, |
| | and 4 |
| | Change from baseline in POEM at Weeks 1, 2, and 4 |
| | Change from baseline in DLQI score at Weeks 1, 2, |
| | and 4 |
| To evaluate the PD profile of | Exploratory PD endpoints: |
| Compound A in blood in subjects | Change in selected biomarkers in PBMCs |
| with moderate to severe AD. | Change in T-cell cytokines in PBMCs |

### Study Design:

This will be a Phase 1, randomized, double-blind, placebo-controlled, sequential SAD/MAD study, with a food-effect arm in healthy subjects and multiple doses in subjects with moderate to severe AD. The study will be divided into two parts:
Part 1 (single center):
   Part 1A: SAD cohorts, with food-effect evaluation.
   Part 1B: MAD cohorts.
Part 2 (multi-center):
   Multiple doses in subjects with AD.

Part 1A and Part 1B might be completed sequentially but with partial overlapping. Part 2 will be initiated after completion of Part 1A and Part 1B.

### Part 1A (SAD)—Cohorts 1 to 7:

Part 1A will consist of 7 cohorts (1 cohort per dose level). Each cohort will include 8 subjects (6 subjects receiving the study drug [Compound A capsules] and 2 receiving matching placebo), for a total of 56 subjects.

A staggered dosing schedule will be used for dosing of each cohort under fasting conditions where two (2) sentinel subjects (1 active and 1 placebo) will be dosed initially, and the remaining subjects dosed at least 24 hours later.

Subjects from Cohort 4 will receive the study drug under both fasting (in a first period) and fed conditions (in a second period). All subjects will be dosed on the same day in the fed period. Safety and PK data through at least 96 hours post-dose from Cohort 5 will be reviewed by the SRC prior to dosing the fed period of Cohort 4. Therefore, the fed period of Cohort 4 could be dosed in parallel of the SAD Cohort 6.

Prior to dosing the next cohort from Part 1A, safety and PK data from at least 7 (out of 8) subjects from the prior cohorts through at least 96 hours post-dose will be reviewed by the SRC. Additionally, a blinded interim analysis of the safety ECG data from Day 1 pre-dose to 24 hours post-dose will be performed by a central ECG laboratory (Calrio, Pittsford, NY) after each cohort, starting with Cohort 2 (considering the low Compound A dose administered to Cohort 1, the interim safety ECG data from Cohort 1 will not be needed prior to dosing of Cohort 2 but may be reviewed after completion of Cohort 2), and will be part of the safety data reviewed by the SRC prior to dosing the next cohorts. If stopping criteria have not been met, the SRC may decide to escalate to the next planned dose level.

### Study Procedures:

A total of 20 blood samples will be collected for PK analysis: pre-dose and 0.25, 0.5, 0.75, 1, 1.5, 2, 3, 4, 5, 6, 8, 10, 12, 16, 24, 36, 48, 72, and 96 hours post-dose. For Cohort 4 that will be dosed also under fed conditions, the same sampling schedule will be used in both periods.

For all Part 1A (SAD) cohorts (except for Cohort 4 Period 2 [fed period]), Compound A and M3 metabolite levels will also be quantified in urine for PK analysis. Urine samples will be collected from Day 1 to Day 5 for the following time intervals: spot pre-dose (within 30 minutes before dosing), 0-4, 4-8, 8-12, 12-24, 24-36, 36-48, 48-72, and 72-96 hours post-dose.

Alcohol breath test, urine cotinine test, and urine drug screen will be done at screening and on Day −1.

Serum pregnancy test (for women) will be performed on Day −1 and urine pregnancy test will be performed at screening and follow-up visit.

A brief physical examination will be performed on Day −1, prior to discharge, and at follow-up visit. A complete physical examination will be performed at screening.

Vital signs (blood pressure [BP], heart rate [HR], respiratory rate [RR] and oral temperature [OT]) will be measured at screening, before dosing and approximately 0.5, 1, 2, 4, 6, 12, 24, 48, 72, and 96 hours post-dose (prior to discharge). Vital signs will also be measured at the follow-up visit.

Safety ECG will be performed at screening, before dosing and approximately 0.5, 1, 2, 3, 4, 6, 12, 24, 72, and 96 hours post-dose (prior to discharge). Safety ECG will also be performed at the follow-up visit.

A continuous ECG recording (Holter) will be performed for all cohorts: on Day 1 from 1 hour prior to dosing until 24 hours after dosing. 12-lead ECGs will be extracted at a central ECG laboratory (Clario, Pittsford, NY) at 3 time points prior to dosing (−45, −30, and −15 minutes) and the following time points, paired with PK samples: 0.5, 1, 1.5, 2, 4, 6, 12, and 24 hours post-dose. The fed period of Cohort 4 will not be included in the continuous ECG recording.

Hematology, biochemistry, coagulation, TSH, and urinalysis will be performed at screening, on Day −1, 24 hours post-dose, prior to discharge, and at the follow-up visit.

### Part 1B (MAD)—Cohorts 8 to 10:

Part 1B will consist of 3 cohorts (1 cohort per dose level). Each cohort will include 11 subjects (9 subjects receiving the study drug [Compound A capsules] and 2 receiving matching placebo daily for 14 consecutive days), for a total of 33 subjects.

Part 1B can be initiated only following review of the safety, tolerability, and PK data up to 96 hours post-dose from SAD Cohort 5. Therefore, MAD Cohort 8 could be dosed in parallel of the SAD Cohort 6.

Prior to dosing the next cohort from Part 1B, safety and PK data from at least 9 (out of 11) subjects from the prior cohorts through at least 96 hours post-last dose will be reviewed by the SRC. If stopping criteria have not been met, the SRC may decide to escalate to the next planned dose level.

### Study Procedures:

A total of 39 blood samples will be collected for PK analysis:
On Day 1: before dosing and 0.25, 0.5, 0.75, 1, 1.5, 2, 3, 4, 5, 6, 8, 10, 12, 16, and 24 hours post-dose.
On Days 11, 12, and 13: before dosing.
On Day 14: before dosing and 0.25, 0.5, 0.75, 1, 1.5, 2, 3, 4, 5, 6, 8, 10, 12, 16, 24, 36, 48, 72, and 96 hours post-dose.

Some modification to the PK sample collection schedule of Part 1B may be done based on the PK results from Part 1 A. Modifications may include shortening or lengthening the total interval of collection by up to 24 hours, revising the timepoints and adding up to 3 more PK samples. If any changes are made to the PK timepoints schedule, these changes will also be reflected in the corresponding ECG Holter extraction timepoints.

Biomarkers assessment in blood will be included in the Part 1B (MAD) cohorts. Blood samples for pharmacodynamic (PD) analysis will be collected before Day 1 dosing and 4 hours post-dose on Day 1 and Day 14.

Alcohol breath test, urine cotinine test, and urine drug screen will be done at screening and on Day −1.

Serum pregnancy test (for women) will be performed on Day −1 and urine pregnancy test will be performed at screening and follow-up visit.

A brief physical examination will be performed on Day −1, prior to discharge, and at follow-up visit. A complete physical examination will be performed at screening.

Vital signs (BP, HR, RR, and OT) will be measured at screening, before Day 1 dosing and approximately 0.5, 1, 2, 4, 6, and 12 hours post-dose, before dosing and approximately 2 hours post-dose on Day 2 to Day 13, and before Day 14 dosing and approximately 0.5, 1, 2, 4, 6, 12, 24, 48, 72, and 96 hours post-dose (prior to discharge). Vital signs will also be measured at the follow-up visit.

Safety ECG will be performed at screening, before Day 1 dosing and approximately 0.5, 1, 2, 3, 4, 6, and 12 hours post-dose, before dosing and approximately 2 hours post-dose on Day 2 to Day 13, and before Day 14 dosing and approximately 0.5, 1, 2, 3, 4, 6, 12, 24, 72, and 96 hours post-dose (prior to discharge). Safety ECG will also be performed at the follow-up visit.

A continuous ECG recording (Holter) will be performed: on Day 1 and Day 14 from 1 hour prior to dosing until 24 hours after dosing. If this part is to be analyzed, as per the investigator, sponsor, and/or Clario's judgement, 12-lead ECGs will be extracted at a central ECG laboratory (Clario, Pittsford, NY) at 3 time points prior to dosing (−45, −30, and −15 minutes) on Day 1 and once prior to dosing on Day 14 and at the following time points, paired with PK samples: 0.5, 1, 1.5, 2, 4, 6, 12, and 24 hours post-dose. ECG data will be stored for later cardiodynamic analysis, as warranted.

Hematology, biochemistry, coagulation, TSH, and urinalysis will be performed at screening, on Day −1, on Days 2, 5, 9, 13, 15, and 18 (prior to discharge), and at the follow-up visit.

### Part 2:

Part 2 of the study will be randomized, double-blind, multi-center, and placebo-controlled to investigate the safety, tolerability, PK, PD, and preliminary efficacy of oral Compound A administered daily for 28 days to subjects with moderate to severe AD.

Part 2 will be initiated after completion of Part 1A and Part 1B, and evaluation of the available safety and PK data by the SRC.

Part 2 will include approximately 40 subjects with moderate to severe AD (defined by a Validated Investigator's Global Assessment for AD [vIGA-AD]score of ‘3’ (moderate) or ‘4’ (severe) and a body surface area [BSA] involved with AD≥10% [excluding scalp, genitals, palms, and soles] at screening and Day 1). All subjects will read and sign an informed consent form prior to any screening procedures being performed. Subjects who meet initial screening requirements will be given a diary to complete the pruritus Numerical Rating Scale (NRS) daily, starting at the screening visit. Subjects who fulfill all the inclusion criteria and none of the exclusion criteria will be accepted into the study. After a screening period of no more than 30 days, and no less than 7 days (to collect pruritus NRS between Day −7 and Day −1 and calculate a weekly average baseline score prior to Day 1), subjects will be randomized (1:1) on Day 1 to receive Compound A or placebo as oral capsule(s) daily for 28 days. Randomization will be stratified according to biopsy consent status (yes/no) at Day 1. The dose of Compound A to be tested in Part 2 should not exceed the highest tolerable dose tested in Part 1B (MAD). The treatment period will be followed by a 2-week follow-up period. For scheduled study visits, subjects will come to the study centers on 6 occasions: screening, Day 1, Day 8, Day 15, Day 29, and Day 43/early termination (ET).

The safety and tolerability of Compound A will be assessed by collecting adverse events (AEs), recording vital signs, performing complete and brief physical examinations and safety ECG, and evaluating clinical laboratory results.

The preliminary efficacy of Compound A will be assessed by vIGA-AD, Eczema Assessment and Severity Index (EASI), SCORing Atopic Dermatitis (SCORAD), BSA, and pruritus NRS. Quality of life will be evaluated using Patient-Oriented Eczema Measure (POEM) and Dermatology Life Quality Index (DLQI).

Pharmacokinetic samples will be collected from all subjects prior to study drug administration on Days 1, 8, and 15, as well as at the end of treatment (EOT) visit on Day 29 (or ET, as applicable). Pharmacodynamic blood samples will also be collected prior to study drug administration on Day 1 and at the EOT visit on Day 29 (or ET, as applicable).

The effect of Compound A on skin biomarkers will be evaluated by collecting skin biopsies and tape stripping:
In a subset of approximately 32 subjects (i.e., approximately 16 subjects per arm) who consented to the biopsy: On Day 1, two 4.5-mm skin biopsies will be collected prior to the study drug administration, one from lesional skin and one from adjacent nonlesional skin. On Day 29, one 4.5-mm skin biopsy will be collected from the same area of the lesional skin (outside the scar of the previous biopsy, at least 1 cm away from the previous scar), even if lesion has cleared.
In all subjects: On Day 1, skin tape strips will be collected prior to the study drug administration from lesional skin and from adjacent nonlesional skin. On Day 29, skin strips will be collected from the same area of lesional skin, even if lesion has cleared.

Adhesive skin strips samples and skin biopsies will be collected from adjacent sites, whenever possible, and preferably from the same lesion.

Medical photographs will be taken at selected sites (optional procedure) and for subjects who consented to the procedure. Photographs of a representative AD lesion will be taken on Days 1 and 29 (or ET, as applicable). The photographs taken will not be used for formal data analysis and are for qualitative purposes only.

### Inclusion/Exclusion Criteria:

### Part 1A and Part 1B:

In order to be eligible to participate in the study, a subject must meet all of the following inclusion criteria and none of the exclusion criteria.

### Inclusion Criteria:

1. Male or female, non-smoker (no use of tobacco or nicotine products within 3 months prior to screening), ≥18 and ≤55 years of age at the time of consent, with a body mass index (BMI) of >18.5 and <30.0 kg/m² and body weight ≥50.0 kg for males and ≥45.0 kg for females.

2. Healthy as defined by:
a. the absence of clinically significant illness or surgery within 4 weeks of dosing.
b. the absence of clinically significant history and condition of neurological, endocrine, cardiovascular, respiratory, hematological, immunological, psychiatric, gastrointestinal, renal, hepatic, and metabolic disease.

3. Female subjects of non-childbearing potential must be:
a. post-menopausal (spontaneous amenorrhea for at least 12 months prior to dosing) with confirmation by documented FSH levels≥40 mIU/mL; or
b. surgically sterile (bilateral oophorectomy, bilateral salpingectomy, hysterectomy, or tubal ligation) at least 3 months prior to dosing.

4. Females of childbearing potential who are sexually active with a non-sterile male partner (sterile male partners are defined as men vasectomized since at least 3 months prior to dosing) must be willing to use one of the following acceptable contraceptive methods throughout the study and for 30 days after (the last) study drug administration:
a. simultaneous use of non-hormonal intrauterine device placed at least 4 weeks prior to (the first) study drug administration, and condom for the male partner;
b. simultaneous use of diaphragm, contraceptive sponge, or cervical cap with intravaginally applied spermicide and male condom for the male partner, started at least 21 days prior to (the first) study drug administration.

5. Male subjects who are not vasectomized for at least 3 months prior to dosing, and who are sexually active with a female partner of childbearing potential must be willing to use one of the following acceptable contraceptive methods from the first study drug administration until at least 90 days after (the last) study drug administration:
a. simultaneous use of a male condom and, for the female partner, hormonal contraceptives or non-hormonal intrauterine device used since at least 4 weeks prior to sexual intercourse;
b. simultaneous use of a male condom and, for the female partner, a diaphragm, contraceptive sponge, or cervical cap with intravaginally applied spermicide.

6. Male subjects (including men who have had a vasectomy) with a pregnant partner must agree to use a condom from (the first) study drug administration until at least 90 days after (the last) study drug administration.

7. Male subjects must be willing not to donate sperm from (the first) study drug administration until 90 days following (the last) study drug administration.

8. Female subjects agree to not have egg retrieval from (the first) study drug administration until 1 month after the last study drug administration.

9. Subject is willing to participate and is capable of giving informed consent. Note: Consent must be obtained prior to any study-related procedures.

10. Subjects must be willing to comply with all study procedures and must be available for the duration of the study.

### Exclusion Criteria:

1. Any clinically significant abnormality at physical examination, clinically significant abnormal laboratory test results or positive test for hepatitis B virus (HBV), hepatitis C virus (HCV), or human immunodeficiency virus (HIV) found during medical screening.

2. Evidence of clinically significant hepatic or renal impairment including alanine aminotransferase (ALT) and aspartate aminotransferase (AST) above 1.5× the upper limit of normal (ULN), total bilirubin 2× above the ULN (total bilirubin accepted up to 2× the ULN if direct bilirubin is within normal limits), or creatinine is above the ULN at screening and Day −1.

3. Positive urine drug screen, alcohol breath test, or urine cotinine test at screening or on Day −1.

4. History of significant allergic reaction (e.g., anaphylaxis, prominent respiratory and skin symptoms) or hypersensitivity to any drug, or history of allergic reaction to any excipient in the formulation.

5. Positive pregnancy test at screening or on Day −1.

6. Any of the following abnormalities on 12-lead safety ECG at screening, confirmed by repeat:
a. PR (PR interval)≥210 msec.
b. QRS (QRS complex)≥120 msec.
c. Fridericia's corrected QT interval (QTcF)≥450 msec.

7. Clinically significant vital sign abnormalities (systolic blood pressure lower than 90 or over 140 mmHg, diastolic blood pressure lower than 50 or over 90 mmHg, or heart rate less than 50 or over 100 bpm) at screening.

8. History of alcohol abuse within 1 year prior to screening or regular use of alcohol within 6 months prior to the screening visit that exceeds 10 units of alcohol per week for women or 15 units of alcohol per week for men [1 unit=150 mL of wine, 360 mL of beer, or 45 mL of 40% alcohol]).

9. History of drug abuse within 1 year prior to screening or recreational use of soft drugs (such as marijuana) within 1 month prior to the screening visit or hard drugs (such as cocaine, phencyclidine [PCP], crack, opioid derivatives including heroin, and amphetamine derivatives) within 3 months prior to screening.

10. Participation in a clinical research study involving the administration of an investigational or marketed drug or device within 30 days prior to first dosing, administration of a biological product in the context of a clinical research study within 90 days prior to dosing, or concomitant participation in an investigational study involving no drug or device administration.

11. Use of medications for the timeframes specified below, with the exception of medications exempted by the investigator on a case-by-case basis because they are judged unlikely to affect the PK profile of the study drug or subject safety (e.g., topical drug products without significant systemic absorption):
a. prescription medications within 14 days prior to the first dosing (or prior to the first dosing of each period [i.e., Fasted and Fed] for Cohort 4 of Part 1A);
b. over-the-counter products and natural health products (including herbal remedies, homeopathic and traditional medicines, probiotics, food supplements such as vitamins, minerals, amino acids, essential fatty acids, and protein supplements used in sports) within 7 days prior to the first dosing (or prior to the first dosing of each period [i.e., Fasted and Fed] for Cohort 4 of Part 1A), with the exception of the occasional use of acetaminophen (up to 2 g daily);
c. use of any type of hormone replacement therapy for 30 days before the first dose;
d. depot injection or implant of any drug within 3 months prior to the first dosing;
e. use of any drugs known to strongly inhibit hepatic metabolism within 30 days prior to the first dosing.

12. Donation of plasma within 7 days prior to dosing or donation or loss of 500 mL or more of whole blood within 8 weeks prior to dosing.

13. History of latent or active tuberculosis (TB) or exposure to endemic areas within 8 weeks prior to QuantiFERON®-TB testing performed at screening.

14. Positive QuantiFERON®-TB test indicating possible TB infection.

15. Immunization with a live or live attenuated vaccine within 1 month prior to the first dosing or planned vaccination during the course of the study and up to 4 weeks or 5 half-lives (of the study product), whichever is longer, after the last study drug administration. Note: COVID-19 vaccination is not allowed within at least 14 days prior to first dosing and throughout the study.

16. History of clinically significant opportunistic infection (e.g., invasive candidiasis or pneumocystis pneumonia).

17. Serious local infection (e.g., cellulitis, abscess) or systemic infection (e.g., septicemia) within 3 months prior to screening.

18. Presence of fever (body temperature>37.6° C.) (e.g., a fever associated with a symptomatic viral or bacterial infection) within 2 weeks prior to the first dosing.

19. Subject is a female who is breastfeeding, pregnant, or who is planning to become pregnant during the study.

20. Subject who participated in a previous cohort of this study.

21. For Part 1A (SAD) Cohort 4 only: Subject with a vegetarian/vegan diet, who does not agree to eat the meals provided while at the clinical site.

22. Any reason which, in the opinion of the investigator, would prevent the subject from participating in the study.

### Part 2:

### Inclusion Criteria:

In order to be eligible to participate in this study, a subject must meet all of the following criteria, either at the screening and Day 1 visits or only at one of the specified visits (screening or Day 1) as noted in the criterion:

1. Male or female, non-smoker (no use of tobacco or nicotine products within 3 months prior to screening), ≥18 and ≤65 years, at the time of consent.

2. Subject has a BMI of 18 and 40 kg/m² at screening.

3. Subject has clinically confirmed diagnosis of active AD, according to the Hanifin and Rajka criteria.

4. Subject has at least a 6-month history of AD and had no significant flares in AD for at least 4 weeks before screening (information obtained from medical chart or subject's physician, or directly from the subject).

5. Subject has moderate to severe AD, as defined by a vIGA-AD score of ‘3’ (moderate) or ‘4’ (severe), at screening and Day 1.

6. Subject has AD covering ≥10% of the BSA (excluding scalp, genitals, palms, and soles) at screening and Day 1.

7. Subject has a history of inadequate response to treatment with topical medications (e.g., corticosteroids, calcineurin inhibitors, etc.) within 1 year before the screening visit (information obtained from medical chart or subject's physician, or directly from the subject) or subjects for whom topical treatments are otherwise medically inadvisable.

8. Subject has been using an emollient (except those containing urea) daily for at least 1 week prior to Day 1 (except on visit day before the visit) and agrees to continue using that same emollient daily at the same frequency (ideally once or twice daily) throughout the study.
Notes:
   On the day of scheduled visit, subjects cannot apply emollient before their scheduled visit.
   Every effort should be made to keep the same emollient throughout the study for the same body region. However, the chosen emollient may differ depending on the body region (e.g., body vs face emollient may be different).

9. For female subject of childbearing potential involved in any sexual intercourse that could lead to pregnancy: the subject must agree to use a highly effective contraceptive method from at least 4 weeks before Day 1 until at least 4 weeks after the last study drug administration. Highly effective contraceptive methods include hormonal contraceptives (e.g., combined oral contraceptive, patch, vaginal ring, injectable, or implant), intrauterine devices or intrauterine systems, vasectomized partner(s) (provided his vasectomy was performed ≥4 months prior to screening), tubal ligation, or double barrier method of contraception (e.g., male condom with cervical cap, male condom with diaphragm, and male condom with contraceptive sponge) in conjunction with spermicide.
Notes:
   Subjects must have been on a stable dose of hormonal contraceptives for at least 4 weeks before Day 1.
   The above list of contraceptive methods does not apply to subjects who are abstinent for at least 4 weeks before Day 1 and will continue to be abstinent from penile-vaginal intercourse throughout the study. The reliability of sexual abstinence needs to be evaluated in relation to the duration of the clinical trial and the preferred and usual lifestyle of the subject. Periodic abstinence (calendar, symptothermal, post-ovulation methods) is not acceptable.
   A female subject of nonchildbearing potential is defined as follows:
      Female subject who has had surgical sterilization (hysterectomy, bilateral oophorectomy, or bilateral salpingectomy)
      Female subject who has had a cessation of menses for at least 12 months prior to the screening visit without an alternative medical cause, and a follicle-stimulating hormone (FSH) test confirming nonchildbearing potential (refer to laboratory reference ranges for confirmatory levels)

10. For a male subject involved in any sexual intercourse that could lead to pregnancy, subject must agree to use one of the highly effective contraceptive methods listed in inclusion criterion #9, from Day 1 until at least 90 days after the last study drug administration. If the female partner of a male subject use any of the hormonal contraceptive methods listed above, this contraceptive method should be used by the female partner from at least 4 weeks before Day 1 until at least 90 days after the last study drug administration.

11. Male subjects must be willing not to donate sperm from Day 1 until 90 days following the last study drug administration.

12. Female subjects agree to not have egg retrieval from Day 1 until 1 month after the last study drug administration.

13. Subject is willing to participate and is capable of giving informed consent. Note: Consent must be obtained prior to any study-related procedures.

14. Subjects must be willing to comply with all study procedures (including use of emollient) and must be available for the duration of the study.

### Exclusion Criteria:

A subject who meets any of the following criteria at the screening and/or Day 1 visits, as applicable, will be excluded from participation in this study:

1. Subject is a female who is breastfeeding, pregnant, or who is planning to become pregnant during the study.

2. Female subject of childbearing potential has had a positive serum pregnancy test at screening or positive urine pregnancy test at Day 1.

3. Subject has clinically infected AD.

4. Subject has a history of skin disease or presence of skin condition that, in the opinion of the investigator, would interfere with the study assessments.

5. Subject is known to have immune deficiency or is immunocompromised.

6. Subject has a history of cancer or lymphoproliferative disease within 5 years prior to Day 1. Subjects with successfully treated nonmetastatic cutaneous squamous cell or basal cell carcinoma and/or localized carcinoma in situ of the cervix are not to be excluded.

7. Subject had a major surgery within 8 weeks prior to Day 1 or has a major surgery planned during the study.

8. Subject has any clinically significant medical condition or physical/laboratory/ECG/vital signs abnormality that would, in the opinion of the investigator, put the subject at undue risk or interfere with interpretation of study results.

9. Subject has positive results for hepatitis B surface antigens (HbsAg), antibodies to hepatitis B core antigens (anti-HBc), HCV, or HIV.

10. Subject has had a positive TB infection test at screening. Subject will be evaluated for latent TB infection with a purified protein derivative (PPD) test or a QuantiFERON®-TB Gold test. Subjects who demonstrate evidence of latent TB infection (either PPD ≥5 mm of induration or positive QuantiFERON®-TB Gold test, irrespective of bacille Calmette-Guérin vaccination status) will not be allowed to participate in the study. Note: Subjects with documented completed treatment for latent TB may be allowed to participate in the study without retesting, as per investigator's judgement.

11. Subject with any of the following laboratory values at the screening visit:
a. Hemoglobin<11.0 g/dL (<110.0 g/L) or hematocrit<30% (<0.30 v/v);
b. White blood cell count<3.0×109/L (<3000/mm³);
c. Absolute neutrophil count of <1.5×109/L (<1500/mm³);
d. Absolute lymphocyte count of <0.5×109/L (<500/mm³);
e. Platelet count<100×10⁹/L;
f. Estimated creatinine clearance<40 mL/min based on the Cockcroft-Gault calculation or serum creatinine value greater than 1.5 times the ULN;
g. AST or ALT values greater than 2 times the ULN.

12. Subject has used dupilumab within 26 weeks prior to Day 1.

13. Subject has used tralokinumab within 12 weeks prior to Day 1.

14. Subject has used doxepin within 1 week prior to Day 1.

15. Subject has used hydroxyzine or diphenhydramine within 1 week prior to Day 1.

16. Subject has used topical products containing urea within 1 week prior to Day 1.

17. Subject has used systemic antibiotics within 2 weeks or topical antibiotics within 1 week prior to Day 1.

18. Subject has used any topical medicated treatment that could affect AD within 2 weeks prior to Day 1, including, but not limited to, topical corticosteroids, crisaborole, calcineurin inhibitors, ruxolitinib, tars, antimicrobials, medical devices, and bleach baths.

19. Subject has used any drugs known to strongly inhibit hepatic metabolism within 4 weeks prior to the first dosing.

20. Subject has used systemic treatments (other than biologics) that could affect AD less than 4 weeks prior to Day 1 (e.g., retinoids, calcineurin inhibitors, methotrexate, cyclosporine, hydroxycarbamide [hydroxyurea], azathioprine, oral/injectable corticosteroids, baricitinib, upadacitinib, abrocitinib). Note: Intranasal corticosteroids and inhaled corticosteroids are allowed. Eyedrops and eardrops containing corticosteroids are also allowed.

21. Subject has received any ultraviolet (UV)-B phototherapy (including tanning beds) or excimer laser within 4 weeks prior to Day 1.

22. Subject has had psoralen-UV-A (PUVA) treatment within 4 weeks prior to Day 1.

23. Subject has received any marketed or investigational biological agent within 12 weeks or 5 half-lives (whichever is longer) prior to Day 1.

24. Subject has received an intravenous immunoglobulin (IVIg) therapy within 12 weeks prior to Day 1.

25. Subject is currently receiving a nonbiological investigational product or device or has received one within 4 weeks prior to Day 1.

26. Subject has had excessive sun exposure, is planning a trip to a sunny climate, or has used tanning booths within 4 weeks prior to Day 1 or is not willing to minimize natural and artificial sunlight exposure during the study. Use of sunscreen products and protective apparel are recommended when sun exposure cannot be avoided.

27. Immunization with a live or live attenuated vaccine within 1 month prior to the first dosing or planned vaccination during the course of the study and up to 4 weeks or 5 half-lives (of the study product), whichever is longer, after the last study drug administration. Note: COVID-19 vaccination is not allowed within at least 2 weeks prior to first dosing and throughout the study.

28. Subject has a known or suspected allergy to Compound A or any component of the investigational product.

29. Subject has a known history of clinically significant drug or alcohol abuse in the last year prior to Day 1.

30. For subject who consents to skin biopsy assessment: subject has a history of an allergic reaction or significant sensitivity to lidocaine or other local anesthetics.

31. For subject who consents to skin biopsy assessment: subject has a history of hypertrophic scarring or keloid formation in scars or suture sites.

For subject who consents to skin biopsy assessment: subject has taken anticoagulant medication, such as heparin, low molecular weight (LMW)-heparin, warfarin, antiplatelets (except low-dose aspirin≤81 mg which will be allowed), within 2 weeks prior to Day 1, or has a contraindication to skin biopsies. Nonsteroidal anti-inflammatory drugs (NSAIDs) will not be considered antiplatelets and will be allowed.

### Statistical Methods:

Continuous variables will be summarized in tables and will include the number of subjects, mean, standard deviation (SD), median, minimum, and maximum. Categorical variables will be presented in tables as frequencies and percentages. A complete description of the statistical analyses to be performed on safety and tolerability data, on the PK and PD data, and on preliminary efficacy data will be presented in Statistical Analysis Plan(s) (SAPs).

### Safety Analysis:

### Part 1 and Part 2:

Safety and tolerability to Compound A will be evaluated through the assessment of AEs, vital signs, ECG, and clinical laboratory parameters. Safety and tolerability data will be reported using descriptive statistics for each treatment group and cohort, separately for each part.

### Part 1 Only:

For the cardiodynamic ECG evaluation, the primary analysis will be based on concentration-corrected QT interval (QTc) modeling of the relationship between the plasma concentrations of Compound A (and potentially its metabolite) and change from baseline QTcF (ΔQTcF) with the intent to exclude an effect of placebo-correctcd ΔQTcF (ΔΔQTcF)>10 msec at clinically relevant Compound A plasma concentrations. In addition, the effect of Compound A on the placebo-corrected ΔQTcF ΔHR, ΔPR, and ΔQRS (ΔΔQTcF, ΔΔHR, ΔΔPR, and ΔΔQRS) will be evaluated at each post-dosing time point (‘by-time point’ analysis) using the Intersection Union Test. An analysis of categorical outliers will be performed for changes in HR, PR, QRS, QTcF, T-wave morphology and U-wave presence.

### Pharmacokinetic Analysis:

### Part 1A (SAD):

Summary statistics will be used to describe the PK profile for each cohort. The power model approach will be performed on the AUC₀₋ₜ, AUC_{0-inf}, and Cₘₐₓ data to investigate the dose-proportionality.

For evaluation of the food-effect in Cohort 4, PK data (In-transformed AUC₀₋ₜ, AUC_{0-inf}, and Cmax) following a high-fat meal versus under fasting conditions will be compared by an analysis of variance (ANOVA) using SAS®. The ratio (fed/fasting) and 90% geometric confidence interval will also be calculated. Intra- and inter-subject coefficient of variation will also be estimated. A non-parametric approach will be used to assess the difference in Tₘₐₓ between the fed and fasted treatment groups.

Summary statistics will be used to describe urinary excretion (Ae₀₋ₜ, Rₘₐₓ, T_{Rmax}, and Cl_{R}) of Compound A and M3 metabolite.

### Part 1B (MAD):

Summary statistics will be used to describe the PK profile for each cohort. The power model approach will be performed on AUC₀₋₂₄, Cₘₐₓ, AUC_{0-τ}, C_{max ss}, AUC₀₋ₜ, and AUC_{0-inf} data to investigate the dose-proportionality.

Helmert's contrasts will be used to determine the time to steady-state.

Comparisons between Day 1 and Day 14 MAD PK parameters: AUC₀₋₂₄ vs AUC_{0-τ}, and Cₘₐₓ vs C_{max ss} will be done by ANOVA. Ratios (Day 14/Day 1) and 90% geometric confidence intervals will be computed for AUC₀₋₂₄ and Cₘₐₓ.

### Part 2:

Compound A and M3 metabolite concentration data will be summarized based on nominal timepoints using descriptive statistics.

### Pharmacodynamic Analysis:

### Part 1 and Part 2:

For PD analyses, changes from baseline in the selected biomarkers in PBMCs will be calculated. Relative changes from baseline in log-scale cytokine levels will also be calculated. These changes from baseline will be summarized and analyzed using statistical methods such as 1-way ANOVA to compare among the different treatment groups. Additional statistical analysis could be done as appropriate.

### Part 2 Only:

Change in skin biomarkers and T-lymphocyte, T-cell and dendritic cell markers will be summarized by treatment group using descriptive statistics.

### Efficacy Analysis:

### Part 2 Only:

Part 2 of this study is not intended to formally evaluate the efficacy of Compound A administered orally for 28 days to subjects with moderate to severe AD. This part, as stated in one of the exploratory objectives, rather intends to evaluate the preliminary efficacy signals of Compound A administered orally for 28 days to subjects with moderate to severe AD.

A Mixed Model for Repeated Measures (MMRM) analysis will be performed for analyzing the change and percent change from baseline in continuous endpoints that are collected in a longitudinal fashion (EASI, vIGA-AD, BSA, SCORAD, and pruritus NRS).

A Chi-square test will be used for preliminary efficacy involving proportion (vIGA-AD, EASI50, EASI75, pruritus NRS).

Descriptive summaries of change from baseline for DLQI and POEM assessments will be presented.

### Sample Size Consideration:

Part 1A (SAD) will consist of 7 cohorts, each including 8 subjects (6 subjects receiving the study drug and 2 receiving matching placebo), for a total of 56 subjects.

Part 1B (MAD) will consist of 3 cohorts, each including 11 subjects (9 subjects receiving the study drug and 2 receiving matching placebo), for a total of 33 subjects.

Depending on safety and tolerability, additional cohorts may be added.

This sample size is not determined based on statistical calculations. A sample size of at least 8 subjects per cohort including 2 subjects on placebo represents a typical panel for a first-in-human (FIH) study and is judged adequate to achieve the objectives of this study. This will expose as few subjects as possible to the treatment while allowing adequate information of the safety, tolerability, and PK of Compound A.

In Part 2 of this study, a sample size of approximately 40 subjects was judged sufficient to characterize the safety, tolerability, PK, PD, and preliminary efficacy of Compound A in subjects with moderate to severe AD. Part 2 is not intended to formally evaluate the efficacy of Compound A administered orally for 28 days to subjects with moderate to severe AD. This part rather intends to investigate any signals of efficacy of Compound A administered orally for 28 days to subjects with moderate to severe AD. Therefore, no formal sample size calculations were performed.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A method of treating an inflammatory disease, disorder, or condition in a patient in need thereof, comprising administering to the patient a therapeutically effective amount of (4-((4-(ethylamino)-3-(trifluoromethyl)-1H-pyrrolo[2,3-b]pyridin-6-yl)amino)-3-methoxyphenyl)(4-morpholinopiperidin-1-yl)methanone (Compound A) or a pharmaceutically acceptable salt or composition thereof.

2. The method of claim 1, wherein the therapeutically effective amount comprises a total daily dose of up to about 600 mg.

3. The method of claim 2, wherein the therapeutically effective amount comprises a total daily dose of about 10 mg, about 30 mg, about 75 mg, about 150 mg, about 300 mg, about 450 mg, or about 600 mg.

4. The method of either of claim 2 or 3, wherein the total daily dose is administered QD.

5. The method of any one of claims 1-4, comprising administering a single dose.

6. The method of any one of claims 1-4, comprising administering at least two doses.

7. The method of any one of claims 1-4, comprising administering Compound A daily for 2-14 consecutive days.

8. The method of any one of claims 1-4, comprising administering daily for 14 consecutive days.

9. The method of any one of claims 2-8, wherein the total daily dose is administered under fasted conditions.

10. The method of any one of claims 2-8, wherein the total daily dose is administered under fed conditions.

11. The method of any one of claims 1-10, wherein the inflammatory disease, disorder, or condition is an autoimmune disease selected from rheumatoid arthritis (RA), ulcerative colitis (UC), Crohn's disease, psoriasis, and atopic dermatitis (AD).

12. The method of any one of claims 1-11, wherein the inflammatory disease, disorder, or condition is RA.

13. The method of any one of claims 1-11, wherein the inflammatory disease, disorder, or condition is UC.

14. The method of any one of claims 1-11, wherein the inflammatory disease, disorder, or condition is Crohn's disease.

15. The method of any one of claims 1-11, wherein the inflammatory disease, disorder, or condition is AD.

16. The method of any one of claims 1-15, wherein administration is oral.

17. The method of any one of claims 2-16, wherein the administration comprises a unit dosage form.

18. The method of any one of claims 2-17, wherein the unit dosage form comprises about 10 mg, about 75 mg, or about 150 mg of Compound A, or pharmaceutically acceptable salt thereof.

19. The method of any one of claims 2-18, wherein the patient does not have one or more of the exclusion criteria as set forth in Example 1.

20. The method of any one of claims 2-19, wherein the patient has one or more of the inclusion criteria as set forth in Example 1.
